(19) United States
(12) Patent Application Publication
(10) Pub. No.: US 2008/0300583 A1
(43) Pub. Date: Dec. 4, 2008

(54) Vascular laser treatment device and method

(75) Inventor: Brian Foley, Wilbraham, MA (US)

Correspondence Address: Dr. Bolesh Skutnik, Esq., 515 Shaker Road, East Longmeadow, MA, 01028, US

(73) Assignee: CeramOptec Industries, Inc.

(21) Appl. No.: 12/156,508

(22) Filed: Jun. 2, 2008

Related U.S. Application Data
(60) Provisional application No. 60/932,601, filed on Jun. 1, 2007.

(51) Int. Cl. A61B 18/20; G02B 6/036

(52) U.S. Cl. 606/15; 385/127

(57) ABSTRACT

An improved device and method for safer and more efficient laser vein treatments are presented. The device includes an optical waveguide optically coupled to a radiation source at its proximal end, having a core, a cladding layer and a tip configured to protect the clad-core, e.g., from contact with collapsing vein walls during laser vein treatment, and to enhance treatment efficiency through improved centering. According to one embodiment, the clad-core is recessed within one or more jacket layers. In some embodiments, the protective jacket on the clad-core may be left on when the jacket layer is added. In embodiments, one or more protective wires are attached to the clad-core or a jacket layer and extend distally past the clad-core. In some such embodiments, three protective wires are substantially equally spaced relative to each other about the circumference of the core, i.e., forming an equilateral triangular pattern. The optical waveguide is useable in conjunction with an introducer structure having protective means to prevent damage to the vein walls, e.g., perforating the vein walls, during insertion of the optical waveguide into the vein. A method of using the device is also disclosed wherein a distal end of the optical waveguide is advanced to a desired position and essentially centered in the vein, and a predetermined wavelength of radiation is output from the distal end of the optical fiber while the optical waveguide is simultaneously withdrawn from the vein.

### CROSS-REFERENCE TO PRIORITY APPLICATION

This application claims priority under 35 U.S.C. § 119 to co-pending U.S. Provisional Patent Application Ser. No. 60/932,601, filed Jun. 01, 2007, entitled “Improved Vascular Laser Treatment Device and Method”, which is hereby expressly incorporated by reference as part of the present disclosure.

### BACKGROUND OF THE INVENTION

1. Field of the Invention

The present invention relates to optical fiber components used in medical laser treatments, and in particular, to fibers for the treatment of veins with laser energy.

2. Invention Disclosure Statement

Underskin laser treatment is an effective method for eliminating many abnormalities, especially skin and vascular problems such as wrinkles and varicose veins, and provides a more proximal access to the area of treatment allowing the use of a less powerful and less harmful laser. Such treatments avoid the need to irradiate through the skin from an external source, which can damage tissue, especially the skin, producing undesired side effects such as external discoloration or scarring. Also, the risk of inadvertent exposure of surrounding tissue to radiation is reduced. Underskin laser treatments can be effective for correcting skin irregularities such as eradicating vascular abnormalities operating in various parts of the body.

One specific application of underskin laser treatments is the correction of vascular abnormalities, such as capillary disorders, spider nevus, hemangioma, and varicose veins. For the treatment of varicose veins, an optical waveguide coupled to a suitable radiation source, is typically positioned in the affected blood vessel. The blood vessel is irradiated to affect the vessel walls and close the vessel. Preferably, the waveguide, typically an optical fiber, is slowly withdrawn during irradiation, to treat and close the blood vessel along a desired length. An exemplary underskin laser treatment device and method is described in U.S. Pat. No. 6,200,332 to Del Giglio, entitled, “Device and Method for Underskin Laser Treatments” having the same assignee as the present invention and which is incorporated by reference herein.

During operation, however, the fiber may be exposed to potentially damaging conditions. For example, as the fiber is withdrawn from the vessel, the vessel closes behind it. Vessel tissue can come in contact with the tip of the fiber, affecting performance of the device and/or causing unwanted damage to the patient, such as perforation of the vein or bruising the patient.

U.S. Application No. 2003/0236517 A1 (Appling et al.) describes an endovascular treatment device including an optical fiber and a protective sleeve, which are axially movable relative to one another. The optical fiber is positioned within the sleeve so that the distal end is in a protected state during insertion of the sleeve into a blood vessel or into a sheath positioned within the blood vessel. After insertion, the sleeve is retracted so that the distal end of the fiber is exposed (operating position) during irradiation. The protective sleeve provides protection to both the distal end of the fiber and the blood vessel or sheath during insertion. Although the protective sleeve may protect the optical fiber while the assembly is inserted into a vessel, once the sleeve is moved to an “operating state” the fiber is exposed and can be damaged.

U.S. Application No. 2004/0010248 A1 (Appling et al.) discloses an endovascular laser treatment device to treat venous diseases such as varicose veins. The device includes a spacer that positions the distal end of the optical fiber away from the inner wall of the blood vessel during delivery of laser energy. For example, a ceramic sleeve can extend over and be spaced radially away from the fiber tip to prevent vessel wall contact. The spacer is used to provide an even distribution of thermal energy around the vessel. The positioning of the spacer, however, still leaves the fiber tip vulnerable to potential contact with vessel tissue during treatment. In addition, there is the possibility of perforation of the vessel, particularly before the vein walls collapse.

In U.S. Application No. 2005/0131400 A1, Hennings et al. disclose an endovascular optical fiber comprising an opaque protective spacer which surrounds the fiber's tip. As an example, a ring shaped polymer extends over the fiber's tip in order to prevent vessel wall contact.

Thus, there is a need to have a device and/or method for underskin radiation treatment wherein damage to the fiber during operation is minimized, or eliminated. The present invention addresses this need.

### OBJECTIVES AND BRIEF SUMMARY OF THE INVENTION

It is an objective of the present invention to provide an improved intravascular laser treatment device with an optical waveguide having a core, a cladding layer and a tip configured to protect the core, for example, from contact with collapsing vein walls during laser vein treatments.

It is another objective of currently preferred embodiments of the present invention to recess the clad-core within one or more jacketing layers to protect the core.

It is still another objective of currently preferred embodiments of the present invention to have a rounded tip for the jacketing layer extending distally past core-clad layer thus avoiding the need for a catheter and/or introducers.

It is yet another objective of currently preferred embodiments of the present invention to employ at least one protective wire structure that extends distally past the core, to guide and protect the core.

It is a further objective of currently preferred embodiments of the present invention to keep the optical waveguide substantially centrally located within the vein during treatment for increased efficiency and safety.

It is still a further objective of the present invention to provide an improved method for intravascular laser treatment using an intravascular laser treatment device, wherein the core of the optical waveguide remains protected for the duration of the treatment.

Briefly stated, the present invention provides an improved device and method for safer and more efficient laser vein treatments. The device includes an optical waveguide optically coupled to a radiation source at its proximal end, having a core, a cladding layer and a tip configured to protect the clad-core, e.g., from contact with collapsing vein walls during laser vein treatment and enhance treatment efficiency through improved centering. According to one exemplary embodiment, the clad-core is recessed within one or more jacket layers. Also in some cases the protective jacket on the clad-core may be left on when the jacket layer is added. In another embodiment, one or more protective wires are attached to the clad-core or a jacket layer and extend distally past the clad-core. For example, three protective wires can be spaced evenly around the circumference of the core, i.e., forming an equilateral triangular pattern. As such, the core remains protected and generally centered for the duration of the treatment. The optical waveguide can be used in conjunction with an introducer structure having a protective means to prevent damage to the vein walls, i.e., perforating the vein walls, during insertion of the optical waveguide into the vein. A method of using the device is also disclosed herein, wherein a distal end of the optical waveguide is advanced to a desired position and essentially centered in the vein. A predetermined wavelength of radiation is output from the distal end of the optical fiber while the optical waveguide is simultaneously withdrawn from the vein. The tip of the optical waveguide protects the clad-core from contact with the collapsing vein wall during withdrawal through the vein.

The above, and other objectives, features and advantages of the present invention and of the currently preferred embodiments thereof will become apparent from the following description read in conjunction with the accompanying drawings, in which like reference numbers in the different figures designate like elements.

### BRIEF DESCRIPTION OF THE DRAWINGS

FIG. 1 is a diagram illustrating an exemplary laser vein treatment device.

FIGS. 2-9 are diagrams illustrating various waveguide tip configurations.

FIG. 10 is a diagram illustrating an exemplary laser vein treatment method.

### DETAILED DESCRIPTION OF PREFERRED EMBODIMENTS

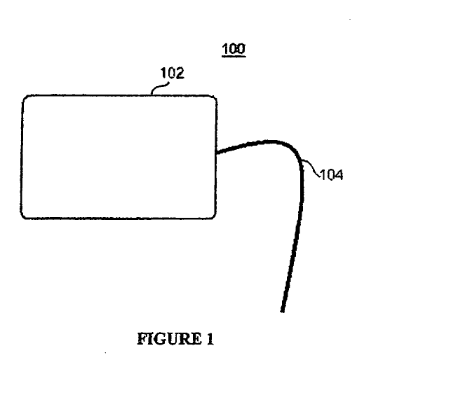

FIG. 1 is a diagram illustrating exemplary laser treatment device 100 for intravascular laser treatment. Laser treatment device 100 includes radiation (laser) source 102 and optical waveguide 104. Optical waveguide 104 may be any device useful for guiding light from a radiation source. A preferred optical waveguide 104 is one or more optical fibers. The optical fiber may be selected from known effective fibers, including generally glass core/glass clad fibers, and fibers having plastic claddings.

Optical waveguide 104 has a proximal and a distal end. The proximal end of optical waveguide 104 is optically coupled to radiation source 102, which may be any known source suitable for emitting radiation of preselected wavelengths and with sufficient power for treatment efficacy. The optimal types of radiation sources and optical waveguides are known in the art for various underskin laser treatments, and are not described further herein. Further, optical waveguide 104 will have a distal tip area as described generally below and in the embodiments presented in FIGS. 2-9. These tips of optical waveguide 104 aid to center the clad-core and enhance the efficiency and are employed to improve safety by preventing contact of the core of optical waveguide 104 with the vein walls during laser vein treatment.

FIGS. 2-9 are diagrams illustrating different tip configurations of an optical waveguide that help center and prevent contact between a radiation-emitting surface, i.e., of the core, of the optical waveguide and vein walls during treatment. Namely, in each of the configurations described below, the tip of the optical waveguide extends distally past, and substantially surrounds, the core of the optical waveguide to protect the core during laser treatment while at the same time leaving an open radiation emitting field. A benefit of the present techniques is that the core of the optical waveguide remains protected and substantially centered throughout the duration of the treatment. This is especially important during laser vein treatments where it is desirable to treat the vein circumferentially in a near uniform manner, generally by heating the blood within the vein, to improve efficiency and wherein the walls of the vein, being subject to treatment, may be continually collapsing around the end of the optical waveguide as the optical waveguide is being withdrawn from the vein.

FIGS. 2A and 2B are side (FIG. 2A) and front view (FIG. 2B) diagrams illustrating an optical waveguide tip configuration having a recessed core. Specifically, optical waveguide 200 comprises clad-core 202 substantially surrounded by an over-layer (or over-tube), i.e., glass jacket layer 204. As shown in FIG. 2A, a distal end of clad-core 202 is recessed within a distal end of glass jacket layer 204. According to an exemplary embodiment, glass jacket layer 204 extends, distally past clad-core 202, a distance d. In the illustrated embodiment, the distance d is about 3 times the clad-core diameter. However, as described further below, the distance d may be varied, and preferably is within the range of about ¼ of the clad-core diameter to not more than about 10 (ten) times the clad-core diameter. Although the distance d may vary based on any of a number of different factors, smaller diameter core fibers may at least in some instances require a greater distance d than larger diameter core fibers. The distance d is optimized to be great enough to prevent collapsing vein walls from contacting the core during treatment, but not too large to significantly interfere with the radiation emitting field, i.e., radiation emitting field 206. In the illustrated embodiment, the glass jacket 204 as over-layer is transparent to the radiation, but non transparent materials may also be used for this first over-layer (jacket) or in additional jackets/over-layers.

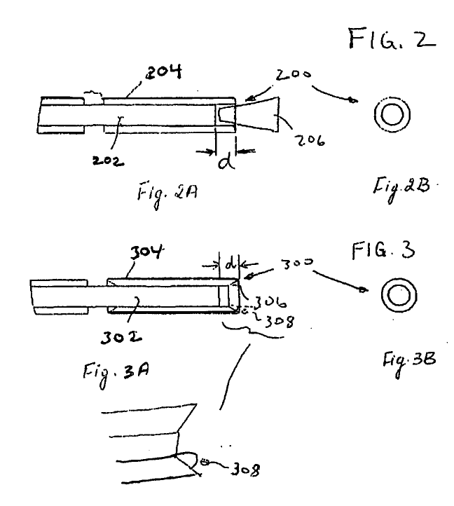

The suggested range for the extension of the over-layer or other tip beyond the distal end of the core-clad face, defining the offset d, is dependent on several parameters of the system. For example, where a number of over-layers, (jackets) are used, so that the overall diameter of the distal end is very much larger than the diameter of the core within the clad-core structure, smaller offsets may be possible. The relatively larger diameter of the over-layers results in improved centering and in effectively protecting the core from the collapsing vessel wall due to the relatively large minimum distance between the outermost edge of the tip to the center area where the core resides. The numerical aperture (“NA”) of the fiber also plays a role in the degree to which the over-layer or other tip extends beyond the distal end of the core-clad face. The range suggested in the example of FIG. 2A above presumes a NA of 0.22 for the clad-core, which is standard for most silica fibers. Other fibers are commercially available either with lower NA values or with higher NA values. To prevent the emitting radiation from striking (or substantially striking) the extended portion of the over-layer(s) (jacket(s)) or other tip configuration a lower NA fiber can have a larger offset relative to the distal end of the over-layer(s) or other tip, whereas a higher NA fiber would require a shorter offset. As shown in FIG. 3A below, the application of beveled distal ends to the over-layers also permits larger offsets as compared to having little or no bevel in the over-layer ends.

As may be recognized by those of ordinary skill in the pertinent art based on the teachings herein, numerous changes may be made to the embodiments shown and described herein without departing from the scope of the invention. For example, the embodiment of FIG. 2A above may define a rounded tip at the distal end of the glass jacket layer 204 thus avoiding the need to use catheters during the procedure. One advantage of such a rounded glass jacket is that it may allow for easier maneuvering in the vein path without the use of a catheter or introducers. In addition, the rounded edge of the glass jacket layer facilitates centering and protecting the core-clad layer from coming in direct contact with the collapsing vein wall and substantially leaving enough open radiation emitting field to effectively treat the vein.

FIGS. 3A and 3B are side (FIG. 3A) and front view (FIG. 3B) diagrams illustrating another optical waveguide tip configuration having a recessed core. Specifically, optical waveguide 300 comprises clad-core 302 substantially surrounded by an over-layer, i.e., glass jacket layer 304. As similarly described, for example, in conjunction with the description of FIG. 2A, above, clad-core 302 is recessed within glass jacket layer 304, a predetermined distance d.

As shown in FIG. 3A, glass jacketing layer 304 has beveled sides 306. According to an exemplary embodiment, sides 306 are beveled at a predetermined angle 308. Beveling sides 306 of glass jacket layer 304 expands the radiation emitting field of optical waveguide 300.

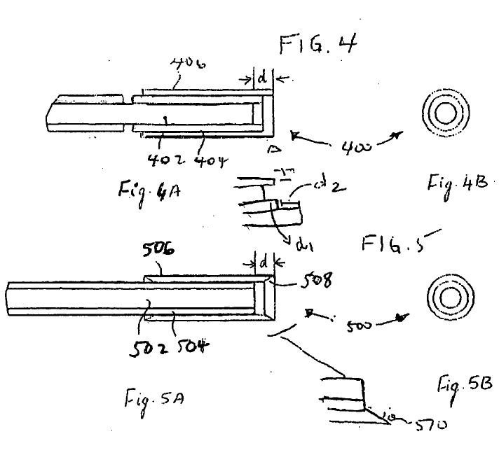

FIGS. 4A and 4B are side (FIG. 4A) and front view (FIG. 4B) diagrams illustrating yet another optical waveguide tip configuration having a recessed core. Specifically, optical waveguide 400 comprises clad-core 402 substantially surrounded by an over-layer, i.e., glass jacket layer 404. Glass jacket layer 404 is in turn substantially surrounded by a second over-layer, i.e., second jacket layer 406. Second jacket layer 406 can be comprised of any suitable jacketing/insulating material, including, but not limited to, glass, ceramic or polymeric materials. As shown in FIG. 4A, clad-core 402 is recessed within glass jacket layer 404, which in turn is recessed within second glass jacketing layer 406. As described, for example, in conjunction with the description of FIG. 2A, above, the clad-core is recessed within the glass jacket layer. Similarly, the glass jacket layer can be recessed within the second glass jacket layer a distance d₂ which may be substantially the same as the initial distance d₁ or may be an added distance as shown. As indicated above, and as shown in FIG. 4A, the total distance d that both glass jacket layers extend distally past the clad-core, is preferably within the range of about ¼ of the clad-core diameter to not more than about 10 (ten) times the clad-core diameter. This stepped configuration of recessing the clad-core within the glass jacket layer, and recessing the glass jacket layer within the second glass jacket layer, helps to maximize the overall distance d that the core is recessed, and thus protected, as well as to maximize the radiation emitting field striking blood in front of the distal end for optimal treatment effects. The multiple over-layers (jackets), can be extended by adding additional over-layers up to the maximum girth permitted by the application.

FIGS. 5A and 5B are side (FIG. 5A) and front view (FIG. 5B) diagrams illustrating still another optical waveguide tip configuration having a recessed core. As with FIG. 4A, described above, two glass jacket layers, i.e., a glass jacket layer and a second glass jacket layer, are present around the clad-core. According to this exemplary embodiment, the second jacket layer is beveled. Specifically, optical waveguide 500 comprises clad-core 502 substantially surrounded by glass jacket layer 504, which in turn is substantially surrounded by second glass jacket layer 506. Second glass jacket layer 506 has beveled sides 508. According to an exemplary embodiment, sides 508 are beveled at an angle 510. Beveling sides 508 of second layer 506 expand the radiation emitting field of optical waveguide 500.

FIGS. 6A and 6B, are side (FIG. 6A) and front view (FIG. 6B) diagrams illustrating an optical waveguide tip configuration having a protective guide wire. Specifically, optical waveguide 600 comprises clad-core 602 substantially surrounded by an over-layer, i.e., glass jacket layer 604. As shown in FIG. 6A, clad-core 602 extends a distance d₃ distally past glass jacket layer 604.

Protective guide wire 606 is attached to, by means such as gluing, soldering, mechanical crimping, etc., and extends distally past, clad-core 602. Protective wire 606 terminates at its distal end in hooked region 608. During laser vein treatment, hooked region 608 prevents the collapsing vein walls from coming into contact with the core, yet does not significantly obstruct the radiation emitting field. Protective guide wire may be made of metal or some other appropriate material. Since it will not displace much blood, its presence has minimal effect on the irradiation step.

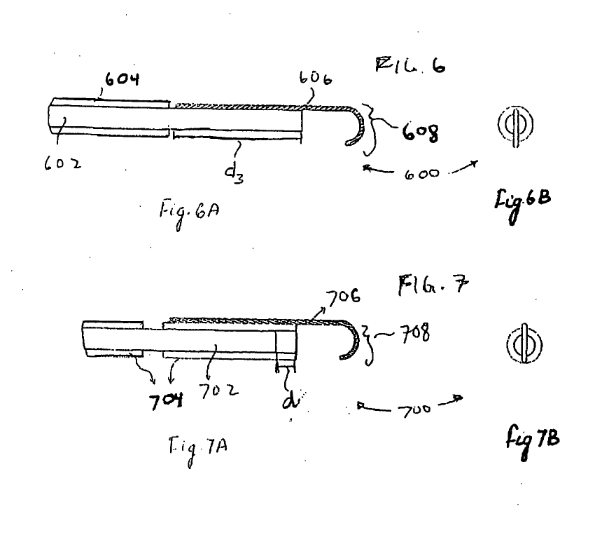

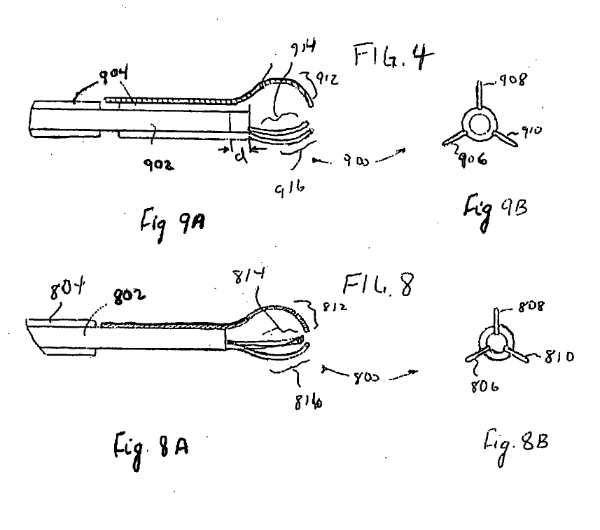

FIGS. 7A and 7B are side (FIG. 7A) and front view (FIG. 7B) diagrams illustrating another optical waveguide tip configuration having a protective wire. Specifically, optical waveguide 700 comprises clad-core 702 substantially surrounded by an over-layer, i.e., glass jacket layer 704. As shown in FIG. 7A, clad-core 702 is recessed within glass jacket layer 704, i.e., glass jacket layer 704 extends distally past clad-core 702 a predetermined distance d.

Protective wire 706 is attached to glass jacketing layer 704 and extends distally past cladding layer/-core 702 and glass jacketing layer 704. As with protective wire 606 described, for example, in conjunction with the description of FIG. 6A, above, protective wire 706 terminates at its distal end in a hooked region, i.e., hooked region 708. Recessing clad-core 702 within glass jacket layer 704 gives an added layer of protection for the core. Further, attaching the protective wire to the glass jacket layer, rather than to the clad-core (see FIG. 6A), enlarges the overall diameter of the tip of the waveguide, thus effectively displacing the walls of the vein at a farther lateral distance from a central axis of the core during treatment and providing additional centering for increased efficiency.

FIGS. 8A and 8B are side (FIG. 8A) and front view (FIG. 8B) diagrams illustrating an optical waveguide tip configuration having multiple protective wires. Specifically, optical waveguide 800 comprises clad-core 802 substantially surrounded by an over-layer, e.g., glass jacket layer 804. As shown in FIG. 8, clad-core 802 extends distally past glass jacket layer 804, (see also description of FIG. 6A, above).

Protective guide/centering wires 806, 808 and 810, as shown in FIG. 8B, are attached to, and extend distally past, clad-core 802. Protective wires 806, 808 and 810 are oriented around the circumference of core 802 so as to form a triangular pattern, e.g., as when viewed from the front. Each of protective wires 806, 808 and 810 terminate at their distal ends in a hooked region, i.e., hooked regions 812, 814 and 816, respectively.

As compared to the protective wires shown, for example, in FIGS. 6 and 7, protective wires 806, 808 and 810 may extend a greater distance laterally from a central axis of the core. Thus, the overall diameter of the tip of the waveguide is enlarged, effectively displacing the walls of the vein at a farther lateral distance from the central axis of the core during treatment. This helps ensure a larger area/volume of blood available in front of the distal end and which can absorb the energy exiting the fiber. By further way of comparison with the protective wires shown in FIGS. 6 and 7, the orientation of protective wires 806, 808 and 810, shown in FIG. 8B around the circumference of the optical waveguide helps to keep the tip of the optical waveguide centrally located within the vein during the treatment, thus ensuring a more even dispersal of the radiation energy and further preventing the collapsing vein walls from contacting the fiber tip. An optimal configuration is to define with the protective wires an equilateral triangle which would enhance centering of the distal end of the fiber continuously as the vein walls are collapsing, while the fiber is withdrawn during treatment.

FIGS. 9A and 9B are side (FIG. 9A) and front view (FIG. 9B) diagrams illustrating another optical waveguide tip configuration having multiple protective wires. Specifically, optical waveguide 900 comprises clad-core 902 substantially surrounded by an over-layer, e.g., glass jacket layer 904. As shown in FIG. 9A, clad-core 902 is recessed within glass jacket layer 904, e.g., glass jacket layer 904 extends distally past clad-core 902 by a predetermined distance d, as in earlier examples.

Protective wires 906, 908 and 910 as shown in FIG. 9B are attached to glass jacket layer 904 and extend distally past clad-core 902 and glass jacket layer 904. Protective wires 906, 908 and 910 are oriented around the circumference of glass jacket layer 904 so as to form a triangular pattern, e.g., as when viewed from the front. Each of protective wires 906, 908 and 910 terminate at their distal ends in a hooked region, i.e., hooked regions 912, 914 and 916, respectively.

Recessing clad-core 902 within glass jacket layer 904 gives an added layer of protection for clad-core 902. Further, attaching the protective wires to the glass jacket layer, rather than to the clad-core (see FIG. 8A), enlarges the overall diameter of the tip of the waveguide, thus effectively displacing the walls of the vein at a farther lateral distance from a central axis of the core and further facilitating centering of the clad-core within the blood vessel for greater efficiency during treatment.

According to an exemplary embodiment, the present laser treatment device and optical waveguide tip configurations can be used as a treatment set in conjunction with a hollow introducer, inside which the waveguide is placed, prior to treatment. Such a treatment set, having a hollow introducer structure, is described, for example, in U.S. patent application Ser. No. 11/800,865, filed by Neuberger et al., entitled “Device and Method for Improved Vascular Laser Treatment,” (hereinafter “Neuberger”), the disclosure of which is incorporated by reference herein. In Neuberger, it is disclosed that a protective means can be positioned on or in a distal end of the introducer, to prevent perforation of a vein during insertion of the waveguide set into the vein.

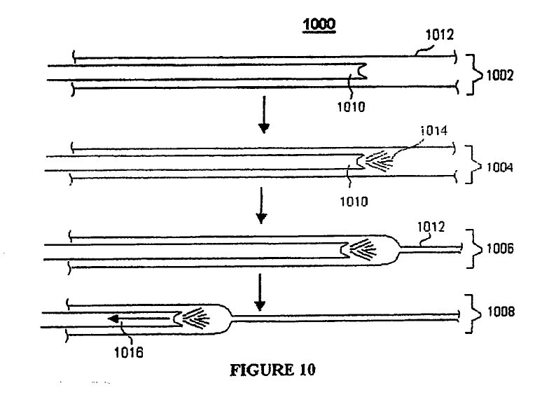

FIG. 10 is a diagram illustrating exemplary laser vein treatment methodology 1000. In step 1002 a laser treatment device, such as exemplary laser treatment device 100 described, for example, in conjunction with the description of FIG. 1, above, is provided having an optical waveguide with one of the waveguide tip configurations described in conjunction with the descriptions of FIGS. 2-9, above. Distal end 1010 of the optical waveguide is advanced to a desired position within vein 1012.

In step 1004, a predetermined wavelength of radiation 1014 is output from distal end 1010 of the optical waveguide. As shown in step 1006, this radiation causes the walls of vein 1012 to collapse. The waveguide tip configuration, in this case a recessed core, prevents the collapsing walls of vein 1012 from contacting the core of the optical waveguide and maintains a more uniform irradiation of the blood and vein walls.

Simultaneously with outputting radiation 1014, distal end 1010 of the optical waveguide is withdrawn from vein 1012, along direction 1016, i.e., back towards a point of entry as shown in step 1008. As described above, an important advantage of the present techniques is that the waveguide tip is configured to protect the core of the optical waveguide for the duration of the treatment of the vein.

As described above, the present laser treatment device and optical waveguide tip configurations may be used as a treatment set in conjunction with an introducer having a protective means. Accordingly, in step 1002, above, the treatment set, i.e., the optical waveguide and the introducer would be advanced to the desired position within the vein. An added step would then be required to expose the optical waveguide tip from the introducer. Several suitable techniques for accomplishing this are described in Neuberger. By way of example only, the optical waveguide can be further advanced within the introducer to pass through the protective means and extend the distal end of the optical waveguide a predetermined distance from an exit opening of the introducer.

Having described preferred embodiments of the invention with reference to the accompanying drawings, it is to be understood that the invention is not limited to the precise embodiments, and that various changes and modifications may be effected therein by one skilled in the art without departing from the scope of the invention as defined in the appended claims.

What is claimed is:

1. An optical waveguide for intravascular laser treatment defining a proximal end, a distal end, a clad-core defining an emitting face at the distal end, at least one over-layer surrounding the clad-core, and a tip extending distally from the emitting face at the distal end, wherein the proximal end of the optical waveguide is configured to be optically coupled to at least one radiation source, the optical waveguide is capable of outputting a predetermined wavelength of radiation from the emitting face of the clad-core, and the tip extends distally from the emitting face of the clad-core a predetermined distance d that facilitates centering the optical waveguide within a blood vessel, protects the emitting face of the clad-core throughout substantially an entire intravascular laser treatment, and maintains an open radiation emitting field in front of the emitting face of the clad-core.

2. An optical waveguide as defined in claim 1, wherein the tip substantially surrounds the emitting face of the clad-core.

3. An optical waveguide as defined in claim 2, wherein the emitting face of the clad-core is recessed within the over layer, and the tip is defined by a distal portion of the over-layer that extends distally from emitting face the distance d and substantially surrounds the emitting face.

4. An optical waveguide as defined in claim 3, wherein the over-layer is a glass jacket layer.

5. An optical waveguide as defined in claim 1, wherein the clad-core defines a diameter, and the distance d is within the range of about ¼ of the clad-core diameter to not more than about 10 (ten) times the clad-core diameter.

6. An optical waveguide as defined in claim 4, wherein the distal portion of the glass jacket over-layer defining the tip is rounded.

7. An optical waveguide as defined in claim 4, wherein an inner edge of the distal portion of the glass jacket over-layer defining the tip is beveled.

8. An optical waveguide as defined in claim 4, further comprising a second glass jacket layer substantially surrounding said glass jacket layer, wherein the second glass jacket layer at least one of (i) substantially surrounds the distal portion of said glass jacket layer, and (ii) substantially surrounds and extends distally beyond the distal portion of said glass jacket layer.

9. An optical waveguide as defined in claim 8, wherein the clad-core defines a diameter, and the second glass jacket layer and said glass jacket layer extend distally from the emitting face of the clad-core a total distance d that is within the range of about ¼ of the clad-core diameter to not more than about 10 (ten) times the clad-core diameter.

10. An optical waveguide as defined in claim 8, wherein an inner edge of the distal portion of the second glass jacket layer defining the tip is beveled.

11. An optical waveguide as defined in claim 1, further comprising at least one protective wire extending distally from the emitting face of the clad-core, and wherein a distal portion of the at least one protective wire defines a substantially hooked shape.

12. An optical waveguide as defined in claim 11, wherein said at least one protective wire is fixedly secured directly to either (i) the clad-core, or (ii) the cover layer surrounding the clad-core.

13. An optical waveguide as defined in claim 12, wherein the cover layer is defined by a glass jacket layer that extends distally from the emitting face of the clad-core a distance d that is within the range of about ¼ of a clad-core diameter to not more than 10 (ten) times the clad-core diameter, and the at least one protective wire is fixedly secured directly to the glass jacket layer.

14. An optical waveguide as defined in claim 11, further comprising three protective wires angularly spaced relative to each other about the circumference of the clad-core in a triangular pattern.

15. An optical waveguide as defined in claim 1, in combination with a radiation source optically coupled to the proximal end of the optical waveguide.

16. An optical waveguide for intravascular laser treatment defining a proximal end configured to be optically coupled to at least one radiation source, a distal end, at least one over-layer surrounding the clad-core, first means for emitting a predetermined wavelength of radiation from the distal end, and second means extending distally from the first means by a predetermined distance d for centering the optical waveguide within a blood vessel, protecting the first means throughout substantially an entire intravascular laser treatment, and maintaining an open radiation emitting field in front of the first means.

17. An optical waveguide as defined in claim 16, wherein the first means is an emitting face of the clad-core, and the second means is at least one of (i) a distal portion of the over-layer extending distally from the emitting face by the predetermined distance d and substantially surrounding the emitting face; (ii) a least one protective wire extending distally from the emitting face by the distance d and defining a distal end portion that is hooked inwardly toward a central axis of the waveguide; (iii) a inner glass jacket layer extending distally from the emitting face, and at least one outer glass jacket layer extending distally from the emitting face and substantially surrounding the inner glass jacket layer, and wherein at least one of the inner and outer glass jacket layers extends distally from the emitting face by the distance d.

18. An optical waveguide as defined in claim 17, wherein the second means comprises a plurality of protective wires substantially equally spaced relatively to each other about the emitting face and extending distally therefrom by the distance d.

19. An optical waveguide as defined in claim 17, wherein the at least one outer glass jacket layer extends distally beyond the inner glass jacket layer, and at least one of the outer and inner glass jacket layers defines an inner beveled annular edge on a distal end thereof.

20. A method for intravascular laser treatment, comprising the steps of:
i. providing an intravascular laser treatment device comprising:
a. a radiation source;
b. a optical waveguide defining a proximal end, a distal end, a clad-core defining an emitting face at the distal end, at least one over-layer surrounding the clad-core, and a tip extending distally from the emitting face at the distal end;
ii. optically coupling the proximal end of the optical waveguide to the radiation source;
iii. advancing the distal end of the optical waveguide to a desired position adjacent to a portion of a vein to be treated;
iv. emitting radiation through the emitting face of the clad-core and into blood within the vein, heating the blood, and in turn thermally damaging the vein;
v. withdrawing the optical waveguide during step (iv); and
vi. using the tip of the optical waveguide throughout steps (iii) through (v) to substantially center the waveguide within the vein, protect the emitting face of the clad-core from contacting the wall of the vein, and maintain an open radiation emitting field in front of the emitting face of the clad-core.

21. The method of claim 16, further comprising the step of:
i. inserting the distal end of the optical waveguide into a hollow introducer including a protective device positioned on or in a distal end of the introducer and forming a treatment set;
ii. advancing the treatment set of the introducer and optical waveguide to the desired position in the vein; and
iii. advancing the optical waveguide within the introducer to pass through the protective device and extend the tip of the optical waveguide a predetermined distance from an exit opening of the introducer.
